(19) United States
(12) Patent Application Publication
(10) Pub. No.: US 2021/0269478 A1
(43) Pub. Date: Sep. 2, 2021

(54) ADJUVANT FOR CELL CULTURE

(71) Applicant: FUKUSHIMA MEDICAL UNIVERSITY, Fukushima-shi, Fukushima (JP)

(72) Inventors: Hideki CHIBA, Fukushima-shi, Fukushima (JP); Kotaro SUGIMOTO, Fukushima-shi, Fukushima (JP)

(73) Assignee: FUKUSHIMA MEDICAL UNIVERSITY, Fukushima-shi, Fukushima (JP)

(21) Appl. No.: 17/053,900

(22) PCT Filed: May 13, 2019
(86) PCT No.: PCT/JP2019/018862

§ 371 Date: Nov. 9, 2020

(30) Foreign Application Priority Data
May 14, 2018 (JP) 2018-093313

(51) Int. Cl. C07K 7/06; C12N 5/00

(52) U.S. Cl. CPC C07K 7/06; C12N 2533/50; C12N 5/0068

(57) ABSTRACT

The present invention provides a component that promotes cell adhesion, cell proliferation, and maintenance of stem cell potential. The invention relates to a peptide containing DPRIEWKKI (SEQ ID NO: 33), or an amino acid sequence obtained by adding, deleting, and/or substituting one amino acid therein. The invention relates to a coating agent for a cell culture substrate, an agent for promoting cell proliferation, an agent for maintaining stem cell potential, or a culture medium containing the peptide or salt thereof. The invention further relates to a cell culture substrate coated with the peptide or salt thereof. The present invention also relates to a method of culturing a cell which uses the coating agent, the cell culture substrate, the agent for promoting cell proliferation, or the agent for maintaining stem cell potential and to a method of producing a cell culture substrate by coating a cell culture substrate with the coating agent.

### TECHNICAL FIELD

The present invention relates to a novel peptide or a salt thereof, a coating agent for a cell culture substrate, an agent for promoting cell proliferation, an agent for maintaining stem cell potential, or a culture medium. The present invention also relates to a cell culture substrate. Further, the present invention relates to a method of culturing a cell, or a method of producing a cell culture substrate.

### BACKGROUND ART

In recent years, the regenerative therapy using stem cells is becoming closer to reality due to remarkable advances in regenerative medicine triggered by the discovery of iPS cells. Clinical tests using stem cells have been initiated for several diseases, and trials of cell therapies for intractable diseases are expected to become even more extensive in future.

A cell therapy using stem cells requires an extremely large amount of cells. In fact, in a model for treating myocardial infarct in a small monkey, as many as one billion cells were grafted into an individual having a body weight of about 10 kg (Non-Patent Literature 1). This is an enormous amount equivalent to 100 or more culture dishes in a size of 10 cm. In addition, since a tissue stem cell generally has a low proliferation rate, it requires a special culture medium containing various small molecules, recombinant proteins, etc. for culturing with sufficient quality being maintained, which is more costly compared to typical cell culture. Therefore, it is desired to establish a new culture method which can dramatically increase the culture efficiency for carrying out a regenerative therapy using stem cells.

Although liquid factors such as a culture medium or additives tend to particularly draw attention in cell culture, optimization of an adhesive substrate is additionally important. In particular, development of an adhesive substrate or a liquid factor which promotes at least one and preferably all of cell adhesion, cell proliferation, and maintenance of stem cell potential is considered to be useful for the development of an efficient culture method.

### CITATION LIST

### Non-Patent Literature

Non-Patent Literature 1: Chong, et al., Nature, 2014, 510, 273-277

### SUMMARY OF INVENTION

### Technical Problem

An object of the present invention is to provide a component which promotes at least one, and preferably all of cell adhesion, cell proliferation, and maintenance of stem cell potential.

### Solution to Problem

The present inventors found that a peptide comprising the amino acid sequence of DPRIEWKKI (SEQ ID NO: 33) adhered to cells. The present inventors also found that a peptide comprising the amino acid sequence of SEQ ID NO: 33 was capable of promoting cell proliferation and maintaining stem cell potential.

The present invention is based at least partly on the above findings and encompasses the following aspects.

(1) A peptide comprising DPRIEWKKI (SEQ ID NO: 33), or an amino acid sequence obtained by adding, deleting, and/or substituting one amino acid in the amino acid sequence of SEQ ID NO: 33, or a salt thereof.

(2) A coating agent for a cell culture substrate, comprising the peptide or salt thereof according to (1).

(3) A cell culture substrate coated with the peptide or salt thereof according to (1).

(4) The coating agent or the cell culture substrate according to (2) or (3) for culturing a stem cell.

(5) An agent for promoting cell proliferation comprising the peptide or salt thereof according to (1).

(6) An agent for maintaining stem cell potential comprising the peptide or salt thereof according to (1).

(7) A culture medium comprising the peptide or salt thereof according to (1).

(8) A method of culturing a cell which uses the peptide or salt thereof according to (1), the coating agent or the cell culture substrate according to any of (2) to (4), the agent for promoting cell proliferation according to (5), the agent for maintaining stem cell potential according to (6), or the culture medium according to (7).

(9) A method of culturing a cell comprising:

a step of coating a cell culture substrate with the peptide or salt thereof according to (1), or the coating agent according to (2) or (4); and

a step of culturing a cell on the coated cell culture substrate.

(10) A method of culturing a cell comprising a step of culturing a cell on the cell culture substrate according to (3) or (4).

(11) A method of culturing a cell comprising a step of culturing a cell in the culture medium according to (7).

(12) The method according to any of (8) to (11), which is a method of culturing a stem cell.

(13) A method of producing a cell culture substrate comprising a step of coating a cell culture substrate with the coating agent according to (2) or (4).

(14) A method of promoting cell proliferation comprising a step of culturing cells in a culture medium comprising the peptide or its salt according to (1).

(15) A method of maintaining stem cell potential comprising a step of culturing a cell in a culture medium comprising the peptide or salt thereof according to (1).

The present specification encompasses the disclosures in Japanese Patent Application No. 2018-093313, which is the basis for the priority of the present application.

### Advantageous Effects of Invention

The present invention can improve the culture efficiency of a cell, particularly a stem cell, by improving at least one and preferably all of cell adhesion, cell proliferation, and maintenance of stem cell potential.

### BRIEF DESCRIPTION OF DRAWINGS

FIG. 1A shows the result of western blotting for cell extracts of ADSCs (adipose-derived stem cells) and the culture supernatant thereof using an antibody recognizing the N-terminus of JAM-C (anti-JAM-C(N) antibody). FIG. 1B shows the result of western blotting using another antibody (anti-JAM-C(C) antibody), the antigen of which is the C-terminus of JAM-C, in addition to the anti-JAM-C(N) antibody. Control represents the results for murine spleen tissues, mFat represents the results for murine adipose tissues, and mADSC represents the results for stem cells derived from murine adipose. N.S. indicates a non-specific signal. FIG. 1C shows the results of the western blotting for the cell extract and the culture supernatant after transiently expressing a JAM-C coding sequence (Jam3-HA) with addition of HA tag at the C-terminus in HEK293T cells. Control shows the results for HEK293T cells in which JAM3-HA was not expressed.

FIG. 2 FIG. 2 shows the results of evaluation of the adhesiveness of a recombinant soluble JAM-C (hereinafter also referred to as rJAM-C) and an ADSC. The rJAM-C, or murine IgG (mIgG) as a negative control (both in 20, 100, and 500 ng (57, 286, and 1,430 ng/cm², respectively)), and type I collagen (500 ng) as a positive control were added to a 96-well plastic culture dish, and then ADSCs were added. After 30 min, the number of adhered cells was measured by crystal violet staining, and the relative absorbance was measured. The absorbance was expressed by a relative absorbance based on the result of the vehicle defined as 1. Error bar indicates standard deviation. Asterisk(s) indicate the result of t-test; where * means P<0.05; and *** means P<0.001.

FIG. 3 shows the results of semi-quantitative RT-PCR, where ADSCs were cultured in a 12-well culture dish coated with rJAM-C (132 ng/cm²), and expression of stem cell markers Klf4, Nanog, c-Myc, and Sox2 were evaluated. The result was expressed as a relative value based on the value of mIgG defined as 1. Error bar indicates standard deviation. Asterisk(s) indicate the result of the t-test; where * means P<0.05; ** means P<0.01; and *** means P<0.001.

FIG. 4 shows the results of evaluation of cell proliferation, where ADSCs were cultured in a 6-well culture dish coated with rJAM-C (104 ng/cm²), the nuclei of cells in a proliferation phase were labelled with BrdU, and the number was divided by the number of DAPI-positive cells. The result was expressed as a relative value based on the value of mIgG defined as 1. Error bar indicates standard deviation. Asterisk(s) indicate the result of t-test; where * means P<0.05; ** means P<0.01; and *** means P<0.001.

FIG. 5 shows the result of an MS spectrum of the peptide obtained in Example 3.

FIG. 6 shows the results of the evaluation of the adhesiveness between JAM-C D1 peptide and F9 cells. JAM-C D1 (286 ng/cm²) was added to a 96-well plastic culture dish, then F9 stem cells were added thereto, and after 30 min the number of adhered cells was measured with crystal violet staining, while performing no coating as a negative control, and using type I collagen (1,430 ng/cm²) and rJAM-C (1,430 ng/cm²) as positive controls. The cell number was expressed as a relative value based on the value of the vehicle defined as 1. Error bar indicates standard deviation. Asterisk(s) indicate the result of the t-test; where * means P<0.05; and *** means P<0.001.

FIG. 7 shows the results of evaluation of cell proliferation, where ADSCs were cultured in a 6-well culture dish coated with JAM-C D1 (52 ng/cm²), the nuclei of the cells in proliferation phase were labelled with BrdU, and the numbers of BrdU-positive cells and negative cells were respectively divided by the number of DAPI-positive cells. The results are shown relative to the total cell number defined as 1. P-value was calculated by a chi-square test (n=353, n=501).

FIG. 8 shows the results of evaluation by semi-quantitative RT-PCR, where ADSCs were cultured in a 6-well culture dish coated with JAM-C D1 (52 ng/cm²), and expression of stem cell markers Nanog, Klf4, Sox2, and c-Myc were evaluated. The result was expressed as a relative value with respect to the value of vehicle defined as 1. Error bar indicates standard deviation in three experiments. Asterisk(s) indicate the result of t-test; where * means P<0.05; ** means P<0.01; and *** means P<0.001.

### DESCRIPTION OF EMBODIMENTS

### <1. Peptide or Salt Thereof>

In an aspect, the present invention relates to a peptide comprising or consisting of DPRIEWKKI (SEQ ID NO: 33), or an amino acid sequence obtained by adding, deleting, and/or substituting one amino acid in the amino acid sequence of SEQ ID NO: 33, or a salt thereof (hereinafter this peptide is also referred to as a “peptide of the present invention”). The amino acid sequence of SEQ ID NO: 33 is a sequence consisting of 9 amino acids comprised in the extracellular domain of JAM-C.

A “salt” means herein a salt prepared using a base or an acid based on a specific substituent (e.g., an amino group or a carboxyl group) of a compound. The salt may be classified into a basic addition salt and an acid addition salt depending on the base or acid that is used.

Examples of a “basic addition salt” include an alkali metal salt, such as a sodium salt and a potassium salt, an alkali earth metal salt, such as a calcium salt and a magnesium salt, an aliphatic amine salt, such as a trimethylamine salt, a triethylamine salt, a dicyclohexylamine salt, an ethanolamine salt, a diethanolamine salt, a triethanolamine salt, and a procaine salt, an aralkyl amine salt, such as N,N-dibenzyl ethylenediamine, a heterocyclic aromatic amine salt, such as a pyridine salt, a picoline salt, a quinoline salt, and an isoquinoline salt, a basic amino acid salt, such as an arginine salt, and a lysine salt, a quaternary ammonium salt, such as a tetramethylammonium salt, a tetraethylammonium salt, a benzyl trimethylammonium salt, a benzyl triethylammonium salt, a benzyl tributylammonium salt, a methyl trioctylammonium salt, and a tetrabutylammonium salt, an ammonium salt, and so forth.

Examples of an “acid addition salt” include an inorganic acid salt, such as a hydrochloride, a sulfate, a nitrate, a phosphate, a carbonate, a hydrogencarbonate, and a perchlorate, an organic acid salt, such as an acetate, a propionate, a lactate, a maleate, a fumarate, a tartrate, a malate, a citrate, and an ascorbate, a sulfonate, such as a methanesulfonate, an isethionate, a benzenesulfonate, and a p-toluenesulfonate, and an acidic amino acid, such as an aspartate, a glutamate, and so forth.

In an embodiment, the peptide of the present invention comprises the extracellular domain of JAM-C. JAM-C means herein Junctional adhesion molecule C. It is known that JAM-C is a protein belonging to the immunoglobulin superfamily, and is expressed in various cells and tissues in humans, such as platelets, T cells, and epithelial cells, and is responsible for a variety of functions, including inflammatory responses (Ebnet, Physiol. Rev., 2017, 97, 1529-1554; and Kummer et al., Cells, 2018, 26, E25).

It has been reported that JAM-C is solubilized by cleavage at a proximal site of the extracellular domain and released out of the cell (Rabquer, et al., J. Immunol., 2010, 185, 1777-1785). JAM-C comprises an IG like-1 domain, an IG like-2 domain, a transmembrane domain, and a PC like domain in the mentioned order from the N-terminal side. The “extracellular domain of JAM-C” means herein an amino acid sequence on the N-terminus side from the transmembrane domain, in other words it may be a peptide comprising the IG like-1 domain and the IG like-2 domain. The extracellular domain of JAM-C may be easily identified by a person skilled in the art by means of a protein domain prediction and/or alignment with a JAM-C of other organism species. The domain prediction can be performed by a public program such as SMART and Pfam. For more precise identification of the extracellular domain of JAM-C, for example, a full-length JAM-C may be recombinantly expressed, and peptides secreted into the culture supernatant may be analyzed.

A peptide of the present invention can be prepared by chemical synthesis, such as solid-phase synthesis and liquid-phase synthesis, according to a conventional method. Further, a peptide of the present invention can also be prepared by a biological method. In this case, a peptide of the present invention may be natural or recombinant. A peptide of the present invention may be obtained by a method that is publicly known to those skilled in the art, for example, by purifying a natural protein from a cell or a tissue, or may be produced by a genetic recombination method. For example, a peptide of the present invention may be prepared by culturing a host cell to which a vector comprising a polynucleotide encoding the peptide of the present invention (or a full-length of JAM-C or the extracellular domain of JAM-C) is introduced, and collecting the cell lysate or the supernatant. When a peptide is obtained by recombination, it may be collected from within the cell or the supernatant. For example, when a peptide of the present invention comprises the extracellular domain of JAM-C, the protein may be obtained from within the cell by expressing only the extracellular domain of JAM-C, or the protein may be obtained by expressing a full-length of JAM-C, solubilizing the protein by cleavage at a proximal site, and obtaining it from the culture supernatant.

The peptide of the present invention may comprise another sequence in addition to an amino acid sequence of SEQ ID NO: 33, or an amino acid sequence obtained by adding, deleting, and/or substituting one amino acid in the amino acid sequence of SEQ ID NO: 33, or the extracellular domain of JAM-C. Examples of such a sequence include a signal peptide which promotes secretion into a culture supernatant, a tag peptide which promotes recovery, purification, or detection as described below, and a sequence of other functional peptides.

A chemically synthesized, natural, or recombinant peptide of the present invention can be collected, or purified by a conventional method. For example, recovery or purification may be performed by any one of, or a combination of two or more of, chromatography, such as gel filtration chromatography, ion-exchange column chromatography, affinity chromatography, reverse phase column chromatography, and HPLC, ammonium sulfate fractionation, ultrafiltration, and immunoadsorption method.

There is no particular restriction on the organism species, from which a peptide of the present invention is derived. Examples thereof include mammals (e.g., primates such as human and rhesus monkey; laboratory animals such as rat, mouse, and Rattus norvegicus; livestock animals such as pig, cattle, horse, sheep, and goat; and pet animals such as dog and cat; preferably human or mouse, and most preferably human), birds (e.g., Gallus domesticus and Gallus gallus), reptiles, amphibians (e.g., Xenopus laevis and Xenopus tropicalis), and fish (zebrafish, etc.). Mammals are preferable.

The amino acid sequence of JAM-C and the polynucleotide sequence of the gene encoding JAM-C are available, for example, from public databases (e.g., NCBI (USA), DDBJ (Japan), EMBL (Europe)) by any method known in the art. For example, human JAM-C may comprise the amino acid sequence of SEQ ID NO: 1, and the polynucleotide encoding JAM-C may comprise the base sequence of SEQ ID NO: 2. Similarly, the JAM-C of rhesus monkey, dog, mouse, Rattus norvegicus, sheep, Gallus gallus, Xenopus tropicalis, and zebrafish may comprise amino acid sequences of SEQ ID NOs: 3, 5, 7, 9, 11, 13, 15, and 17, respectively. Further, the polynucleotides encoding JAM-C of rhesus monkey, dog, mouse, Rattus norvegicus, sheep, Gallus gallus, Xenopus tropicalis, and zebrafish may comprise amino acid sequences of SEQ ID NOs: 4, 6, 8, 10, 12, 14, 16, and 18, respectively. In this regard, the amino acid sequence of SEQ ID NO: 33 is conserved among all of JAM-Cs of rhesus monkey, dog, mouse, Rattus norvegicus, sheep, Gallus gallus, and Xenopus tropicalis.

The extracellular domain in the above amino acid sequence of JAM-C can be easily identified by a person skilled in the art by means of a protein domain prediction and/or alignment with JAM-C of other organism species as described above. For example, the extracellular domain of JAM-C may be a peptide comprising:

(i) an amino acid sequence from position 1 to 244 of any of SEQ ID NOs: 1, 3, 5, 7, and 9, an amino acid sequence from position 1 to 263 of SEQ ID NO: 11, an amino acid sequence from position 1 to 234 of SEQ ID NO: 13, an amino acid sequence from position 1 to 233 of SEQ ID NO: 15, an amino acid sequence from position 1 to 236 of SEQ ID NO: 17, preferably an amino acid sequence from position 1 to 244 of any of SEQ ID NOs: 1, 3, 5, 7, and 9, and most preferably an amino acid sequence from position 1 to 244 of SEQ ID NO: 1;

(ii) an amino acid sequence having an identity of 90% or higher to any of the amino acid sequences of (i); or

(iii) an amino acid sequence obtained by adding, deleting, and/or substituting one or several amino acids in any of the amino acid sequences of (i).

The range of “one or several” with respect to an amino acid sequence is herein from 1 to 10, preferably from 1 to 7, and more preferably from 1 to 5, for example, from 1 to 4, from 1 to 3, or 1 or 2.

There is no particular restriction on the amino acid length of a peptide of the present invention, and it may be, for example, 8 or more amino acids, 9 or more amino acids, or 10 or more amino acids, and, for example, 300 or less, 250 or less, 200 or less, 150 or less, and preferably 100 or less, 50 or less, 40 or less, 30 or less, 20 or less, 15 or less, or 10 or less.

When a peptide of the present invention comprises the extracellular domain of JAM-C, the amino acid length thereof is not particularly limited, and it may be, for example, 150 amino acids or more, 200 amino acids or more, 220 amino acids or more, or 240 amino acids or more, and 1000 amino acids or less, 500 amino acids or less, 400 amino acids or less, or 300 amino acids or less.

Since the amino acid length of a peptide of the present invention can be as short as from 10 to several hundreds, it can have an advantage of low production cost compared to a polypeptide such as laminin having an amino acid length as long as several thousands.

In an embodiment, a peptide of the present invention promotes at least one and preferably all of cell adhesion, cell proliferation, and maintenance of stem cell potential.

### <2. A Coating Agent, a Cell Culture Substrate, an Agent for Promoting Cell Proliferation, and an Agent for Maintaining Stem Cell Potential>

In an aspect, the present invention relates to a coating agent for a cell culture substrate comprising the peptide or salt thereof described in <1. Peptide or salt thereof>.

There is herein no particular restriction on the “cell culture substrate”, insofar as it is used in culturing cells. Examples thereof include a cell culture dish, a cell culture bottle (or flask), a multi-well plate, and microcarriers. A commercially available product may be used as a cell culture substrate. Also, there is no particular restriction on the material of a culture substrate, and examples thereof include glass or plastic. When a coating agent comprising a peptide or a salt thereof of the present invention is coated on a cell culture substrate, at least one and preferably all of cell adhesion property, cell proliferation property, and property of maintenance of stem cell potential of the cell culture substrate can be improved.

In an aspect, the present invention relates to a cell culture substrate coated with a peptide or a salt thereof of the present invention. “Coat(ing)” means herein a step of treating and covering at least a portion of the surface of a cell culture substrate. There is no particular restriction on a method of coating, and, for example, it may be performed by adding an appropriate concentration of a peptide or a salt thereof of the present invention to a cell culture substrate, leaving it, and if necessary, washing it once or more times with a solution such as PBS or water. There is no particular restriction on the concentration at which the peptide or salt thereof is added to the cell culture substrate, and it may be, for example, 0.04 μg/mL or more, 0.1 μg/mL or more, 0.2 μg/mL or more, or 0.4 μg/mL or more, and may be 100 μg/mL or less, 50 μg/mL or less, 20 μg/mL or less, or 10 μg/mL or less. Further, the concentration at which the peptide or salt thereof is added to the cell culture substrate may be, for example, 0.5 ng/cm² or more, 5 ng/cm² or more, 10 ng/cm² or more, 25 ng/cm² or more, or 50 ng/cm² or more, and may be 150 μg/cm² or less, 15 μg/cm² or less, 6 μg/cm² or less, 3 μg/cm² or less, or 1.4 μg/cm² or less. Also, there is no restriction on the time of leaving after the addition of the peptide or salt thereof, and it may be, for example, 1 hour or more, 3 hours or more, 6 hours or more, or 9 hours or more, and may be 120 hours or less, 48 hours or less, 24 hours or less, 18 hours or less, and preferably about 12 hours. There is no particular restriction on the temperature after the addition of the peptide or salt thereof, and it may be, for example, from 20° C. to 40° C., from 25° C. to 35° C., and about 30° C. or room temperature.

In an embodiment, a coating agent or a cell culture substrate of the present invention is applied to a cell expressing JAM-C and/or JAM-B (Junctional Adhesion Molecule B).

In an embodiment, a coating agent or a cell culture substrate of the present invention is for culturing a stem cell. The stem cell may be a cell population solely consisting of stem cells, or a cell population comprising stem cells abundantly. A “stem cell” means herein a cell that has both the ability to differentiate into a different cell type or various cell types, and the ability of self-replication. Examples of a stem cell include a cell in an undifferentiated state in a living tissue, such as bone marrow, blood, skin, and fat (collectively referred to as somatic stem cells, and examples thereof include Muse cell), an embryonic stem cell (ES cell), and an induced pluripotent stem cell (iPS cell). Such a stem cell can be produced by a publicly known method, or it can also be obtained from a particular institution, or can be purchased as a commercial product. These stem cells may be any of a primary culture cell, a subcultured cell, and a frozen cell.

There is no restriction on the origin of a cell herein, and examples thereof include mammals (e.g., primates such as human and rhesus monkey; laboratory animals such as rat, mouse, and Rattus norvegicus; livestock animals such as pig, cattle, horse, sheep, and goat; and pet animals such as dog and cat; preferably human or mouse, and most preferably human), birds (e.g., Gallus domesticus and Gallus gallus), reptiles, amphibians (e.g., Xenopus laevis and Xenopus tropicalis), and fishes (zebrafish, etc.). Mammals are preferable.

In an aspect, the present invention relates to an agent for promoting cell proliferation, or an agent for maintaining stem cell potential, comprising a peptide or a salt thereof of the present invention. The peptide or salt thereof of the present invention and the cell are as described above.

The degree of cell proliferation by an agent for promoting cell proliferation may be not less than 1.1-fold, 1.2-fold, 1.3-fold, 1.4-fold, 1.5-fold, 1.6-fold, 1.7-fold, 1.8-fold, 1.9-fold, or 2-fold, compared to a case where a peptide or a salt thereof of the present invention is not used. The degree of cell proliferation can be measured, for example, by the method described in Example 2.

The “stem cell potential” refers to both the ability to differentiate into another cell, and the ability of self-replication, and maintenance of stem cell potential refers maintaining or improving these abilities. The stem cell potential can be measured by the expression level of a stem cell marker, such as Kfl4, c-Myc, Nanog, and Sox2, and a higher expression level of these markers is considered to indicate that the stem cell potential is maintained or improved. A stem cell marker can be measured, for example, by the method described in Example 2.

Without being bound by a theory, cell proliferation and maintenance of stem cell potential are considered to be provided (i) by an increase in cell adhesiveness, and/or, (ii) when the extracellular domain of JAM-C associates with JAM-B, and a Src family kinase (SFK) recruited by the complex phosphorylates JAM-B, which further has a downstream signaling pathway.

The coating agent, the agent for promoting cell proliferation, and the agent for maintaining stem cell potential of the present invention may consist of a peptide or a salt thereof of the present invention, or may additionally comprise another component. Such another component is not limited, and examples thereof include a buffer solution, a surfactant, and a stabilizer, as well as another coating agent, another agent for promoting cell proliferation, and another agent for maintaining stem cell potential.

In an aspect, the present invention relates to a culture medium comprising a peptide or a salt thereof of the present invention. The culture medium of the present invention can be easily prepared by adding a peptide or a salt thereof of the present invention to an ordinary culture medium. The type of the culture medium is not limited, and it may be, for example, a commercially available culture medium (e.g., DMEM, MEM, BME, RPMI 1640, F-10, F-12, DMEM-F12, α-MEM, IMDM, and MacCoy's 5A culture medium), or a prepared culture medium. The concentration of a peptide in a culture medium may be, for example, 0.04 μg/mL or more, 0.1 μg/mL or more, 0.2 μg/mL or more, or 0.4 μg/mL or more, and may be 100 μg/mL or less, 50 μg/mL or less, 20 μg/mL or less, or 10 μg/mL or less.

### <3. Method of Culturing a Cell>

In an aspect, the present invention relates to a method of culturing a cell which uses a peptide, or a salt thereof as described in <1. Peptide or salt thereof>, a coating agent, a cell culture substrate, an agent for promoting cell proliferation, an agent for maintaining stem cell potential, or a culture medium as described in <2. A coating agent, a cell culture substrate, an agent for promoting cell proliferation, and an agent for maintaining stem cell potential>.

In an aspect, the present invention relates to a method of culturing a cell comprising a step of coating a cell culture substrate with a peptide or a salt thereof as described in <1. Peptide or salt thereof>, or a coating agent as described in <2. A coating agent, a cell culture substrate, an agent for promoting cell proliferation, and an agent for maintaining stem cell potential>, and a step of culturing a cell on the coated cell culture substrate.

Although the coating step is not limited, it may be performed, for example, by adding an appropriate concentration of a peptide or a salt thereof of the present invention, or a coating agent, to a cell culture substrate, leaving it, and, if necessary, washing it once or more times with a solution such as PBS or water. The concentration at which the peptide or salt thereof is added to a cell culture substrate is not limited, and it may be, for example, 0.04 μg/mL or more, 0.1 μg/mL or more, 0.2 μg/mL or more, or 0.4 μg/mL or more, and may be 100 μg/mL or less, 50 μg/mL or less, 20 μg/mL or less, or 10 μg/mL or less. Further, the concentration at which the peptide or salt thereof is added to a cell culture substrate may be, for example, 0.5 ng/cm² or more, 5 ng/cm² or more, 10 ng/cm² or more, 25 ng/cm² or more, or 50 ng/cm² or more, and may be 150 μg/cm² or less, 15 μg/cm² or less, 6 μg/cm² or less, 3 μg/cm² or less, or 1.4 μg/cm² or less. Further, the time of leaving after the addition of the peptide or salt thereof is not limited, and it may be, for example, 1 hour or more, 3 hours or more, 6 hours or more, or 9 hours or more, and may be 120 hours or less, 48 hours or less, 24 hours or less, or 18 hours or less, and preferably about 12 hours. The temperature after the addition of the peptide or salt thereof is also not limited, and it may be, for example, from 20° C. to 40° C., from 25° C. to 35° C., or about 30° C. or room temperature.

The culturing condition in the step of culturing a cell is not limited, and, for example, the culture temperature may be from about 30° C. to about 40° C., and the CO₂ concentration may be from about 2% to about 10%. The culture may be adherent culture or suspension culture, but is preferably adherent culture. The culture medium in the culture step is also not limited, and it may be performed using a commercially available culture medium (e.g., DMEM, MEM, BME, RPMI 1640, F-10, F-12, DMEM-F12, α-MEM, IMDM, and a MacCoy's 5A culture medium), or a prepared culture medium. The duration of the culture step may be, for example, several hours to several days, or may be several days to several weeks or several months after expansion culture or subculture.

In an aspect, the present invention relates to a method of culturing a cell comprising a step of culturing a cell on the cell culture substrate as described in <2. A coating agent, a cell culture substrate, an agent for promoting cell proliferation, and an agent for maintaining stem cell potential>.

Also, in an aspect, the present invention relates to a method of culturing a cell, comprising a step of culturing a cell in a culture medium comprising a peptide or a salt thereof of the present invention. The present method can be applied both to adherent cells and to suspended cells.

The method of culturing a cell of the present invention can have an advantage of promoting cell proliferation and/or maintaining stem cell potential.

In an aspect, the present invention relates to a method of promoting cell proliferation, or a method of maintaining stem cell potential comprising a step of culturing a cell in a culture medium comprising a peptide or a salt thereof of the present invention. The present method can be applied both to adherent cells and to suspended cells.

### <4. Method of Producing a Cell Culture Substrate>

In an aspect, the present invention relates to a method of producing a cell culture substrate comprising a step of coating a cell culture substrate with the coating agent as described in <2. A coating agent, a cell culture substrate, an agent for promoting cell proliferation, and an agent for maintaining stem cell potential>.

Since the coating step is as described in <3. Method of culturing a cell>, the description is omitted here.

### EXAMPLES

### Example 1: Confirmation of Expression

It has been reported that JAM-C is solubilized by cleavage at a proximal site of the extracellular domain and released out of the cell (Rabquer, et al., J. Immunol., 2010, 185, 1777-1785). Therefore, it was examined whether JAM-C is similarly cleaved and solubilized in cultured ADSCs (adipose-derived stem cells).

### (Material and Method)

### Antibody

JAM-C(N) (R&D Systems, AF1213), and JAM-C(C) (Thermo Fisher Scientific, 40-9000) were used for western blotting.

### Isolation of Murine ADSCs

Isolation of murine ADSCs was performed according to a commonly used established method (Lin, et al., Stem Cells Dev. 2008, 17, 1053-63), with the approval of the animal care and use committee of Fukushima Medical University, and in compliance with the Regulations for Care and Use of Laboratory Animals at Fukushima Medical University.

First, subcutaneous adipose tissues in the thigh were collected from 8 to 12 week-old C57BL/6N strain male mice, and shredded with scissors while washing sufficiently with PBS, which were then shaken at 37° C. for 45 min with 0.075% collagenase A (Roche). After removal of floating adipose tissue clumps, the suspension was centrifuged at 200G to isolate an SVF (stromal vascular fraction), and 12×10⁴ cells were inoculated onto a 6 cm culture dish.

### Cell Culture

ADSCs were cultured in a Dulbecco's Modified Eagle Medium (DMEM) supplemented with 10% bovine serum. The culture medium was replaced every 2 days, and when the cell density reached 50 to 70%, cells were detached using a 0.25% trypsin/1 mM EDTA solution, and 2×10⁵ cells were subcultured. Cells at three to five passages were used for analysis.

HEK293T was also cultured in the same method using the same culture medium. Transfection was performed using PEI: polyethylenimine MAX (Cosmo Bio Co., Ltd.) according to the manufacturer's protocol, and the cell extract and the culture supernatant were collected after 48 hours.

### Expression Vector Plasmid

The gene (Jam3) encoding a full-length murine JAM-C PCR-amplified using a cDNA library obtained from the murine kidneys as a template was cloned together with a puromycin resistance gene by a two fragments insertion method using the In-Fusion© HD Cloning Kit (Takara Bio Inc.) at a multiple cloning site (MCS) of the CSII-EF-Venus provided by RIKEN BioResource Center. The primers used were 5′-GAGAATTCTGCAGCGGCCGCCATGGCGCTGAGCCGGCGGCT-3′ (SEQ ID NO: 19), and 5′-CTGCATAGTCCGGGACGTCATACGGATAGCCCGCATAGTCAGGAACATCGTATGG GTAGATAACAAAGGACGATTTGTGTC-3′ (SEQ ID NO: 20) for the amplification of Jam3; and 5′-ATGACGTCCCGGACTATGCAGGATCCTATCCATATGACGTTCCAGATTACGCTGCT ACTAACTTCAGCCTGCTGAAGCA-3′ (SEQ ID NO: 21), and 5′-GGGAGAGGGGCGGATCCTAGGCACCGGGCTTGCGGGTCAT-3′ (SEQ ID NO: 22) for the amplification of the puromycin resistance gene, respectively.

### Western Blotting

Western blotting was performed using a common method previously described (Sugimoto, et al., PLoS One, 2013, 10: e751062013). For protein sampling from cell extracts, a solubilizing solution prepared by adding a protease inhibitor (Complete mini EDTA-free; Roche Diagnostics, Mannheim, Germany), 5 mM NaF, 1 mM Na₃VO₄, and a 1 mM Phenyl methanesulfonyl fluoride Solution (PMFS) to a Radio Immunoprecipitation assay (RIPA) Buffer (25 mM Tris-HCl pH 7.6, 150 mM NaCl, 1% NP-40, 1% sodium deoxycholate, 0.1% SDS) was used. The culture supernatant was concentrated with 10 times the amount thereof of a cooled 10% trichloroacetic acid acetone solution, and then used. SDS-PAGE, transfer, and antibody reaction were performed sequentially, followed by chemiluminescence with ECL Prime (GE Healthcare), which was imaged using a CCD imager Image Quant LAS4000 (GE Health care).

### (Results)

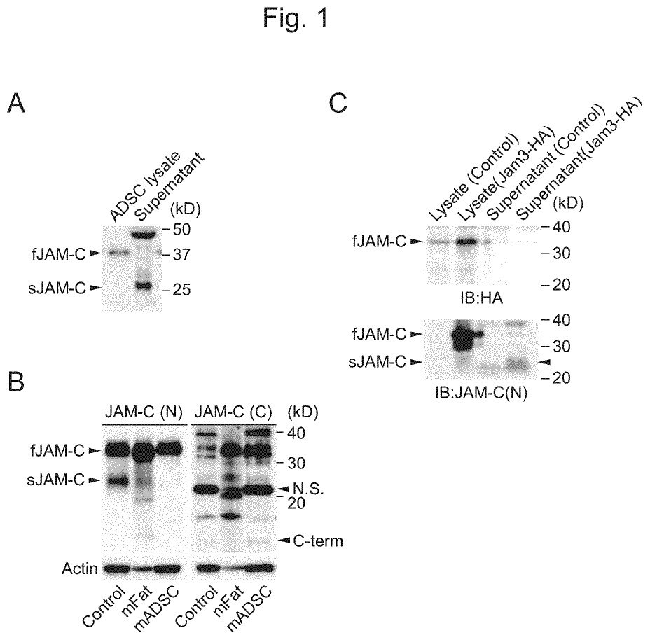

First, the cell extracts from cultured ADSCs and the culture supernatant thereof were subjected to western blotting using an antibody (anti-JAM-C(N) antibody) which recognizes the N-terminus of JAM-C. As a result, a molecule of a full length of approximately 38 kDa (full-length JAM-C (hereinafter also referred to as fJAM-C)) was detected in the cell extracts, and further a smaller molecule of approximately 28 kDa was detected in the culture supernatant (FIG. 1A). This revealed that JAM-C was released as a soluble molecule (soluble JAM-C (hereinafter also referred to as sJAM-C)) into the culture medium.

Next, western blotting for a murine adipose tissue was performed using another antibody (anti-JAM-C(C) antibody) recognizing the C-terminus of JAM-C as the antigen. As a result, a low molecular weight band corresponding to sJAM-C was detected in the spleen and adipose tissues with the anti-JAM-C(N) antibody, but not with the anti-JAM-C(C) antibody (FIG. 1B).

Further, a JAM-C coding sequence (Jam3-HA) to which HA tag was added at the C-terminus was transiently expressed in HEK293T cells, and the cell extracts, and the culture supernatant were similarly subjected to western blotting. As a result, the anti-JAM-C(N) antibody recognized both fJAM-C and sJAM-C in the same manner as described above, but the anti-HA antibody labelled only fJAM-C (FIG. 1C). Thus, it has been shown that sJAM-C is the extracellular domain of JAM-C and is a solubilized body that is released by cleavage. These results showed that the extracellular domain present on the N-terminus side of JAM-C is released as a soluble sJAM-C by cleavage in the vicinity of the transmembrane region.

### Example 2: Effect on Cells

Next, the effect on ADSCs by the sJAM-C deposited on a substrate was examined.

### (Material and Method)

The basic cell culture method and so forth were in accordance with Example 1.

### Cell Adhesion Assay

For each of recombinant soluble JAM-C (polypeptide obtained by linking the Fc region of human IgG1 to the C-terminus of the extracellular domain (Met 1-Asn 241) of human JAM-C; Sino Biological) and murine IgG (GE Healthcare), 20, 100, or 500 ng volume thereof was diluted in 50 μL PBS and disposed into a 96-well round-bottom plate, which was then left standing at 30° C. for 12 hours. As a positive control, 500 ng of collagen Cellmatrix type I-A (Nitta Gelatin Inc.) was processed in the same manner. Then the samples were washed twice with PBS, and, after addition of 100 μL of 10% bovine serum albumin (BSA; Wako Pure Chemical Industries, Ltd.), left standing at 37° C. for 2 hours before blocking.

After washing twice with PBS, ADSCs were detached using a 0.25% trypsin/1 mM EDTA solution and inoculated to the above plate and cultured in a CO₂ incubator at 37° C. for 30 min. Then the culture supernatant was removed, staining was performed with crystal violet, and thereafter washing was performed 10 times with PBS. Finally, 50 μL of 5% SDS was used for lysis, and the absorbance was measured at 560 nm wavelength.

### RNA Extraction and RT-PCR

RNA extraction and RT-PCR were performed using a common method previously described (Sugimoto, et al., PLoS One, 2013, 10: e751062013). The cells were inoculated in a 12-well culture dish in the same manner as in the above cell adhesion assay (the recombinant soluble JAM-C and so forth were used as much as 4 times according to the bottom area), and then cultured in a 10% FBS supplemented DMEM for 48 hours. From these cells RNAs were extracted with a TRIzol Reagent (Ambion), a reverse transcription reaction was carried out using a PrimeScript II 1st strand cDNA Synthesis Kit (Takara Bio Inc.) to obtain a cDNA library. Semi-quantitative PCR was performed using the GoTaq Green Master Mix Kit (Promega) and primers [Sox2 (5′-TAGAGCTAGACTCCGGGCGATGA-3′ (SEQ ID NO: 23), and 5′-TTGCCTTAAACAAGACCACGAAA-3′ (SEQ ID NO: 24)); c-Myc (5′-TGACCTAACTCGAGGAGGAGCTGGAATC-3′ (SEQ ID NO: 25), and 5′-AAGTTTGAGGCAGTTAAAATTATGGCTGAAGC-3′ (SEQ ID NO: 26)); Nanog (5′-AGGGTCTGCTACTGAGATGCTCTG-3′ (SEQ ID NO: 27), and 5′-CAACCACTGGTTTTTCTGCCACCG-3′ (SEQ ID NO: 28)); Klf4 (5′-GCGAACTCACACAGGCGAGAAACC-3′ (SEQ ID NO: 29), and 5′-TCGCTTCCTCTTCCTCCGACACA-3′ (SEQ ID NO: 30)); and Gapdh (5′-ACCACAGTCCATGCCATCAC-3′ (SEQ ID NO: 31), and 5′-TCCACCACCCTGTTGCTGTA-3′ (SEQ ID NO: 32))] to amplify the target sequences. Then, the samples were subjected to electrophoresis in a 2% agarose gel, which were then stained with ethidium bromide and imaged using a CCD imager Image Quant LAS4000 (GE Health care). Then the brightness in a region corresponding to the molecular weight of interest was quantified using the ImageJ (National Institute of Health (NIH)) and statistically analyzed.

### Cell Proliferation Assay

Cells (4×10⁴ cells) were inoculated in a 6-well culture dish, and, 24 hours later, treated with 5-bromo-2′-deoxyuridine (BrdU; #B-5002, Sigma-Aldrich) at the final concentration of 10 μM for 2 hours, and then fixed with 4% PFA. Then, the sample was stained according to the method recommended by the manufacturer, and imaged with a fluorescent phase-contrast microscope (OLYMPUS IX71, Olympus Corporation) and a DP Controller. The number of BrdU-positive particles was divided by that of DAPI-positive ones to obtain a proliferated cell number.

### (Results)

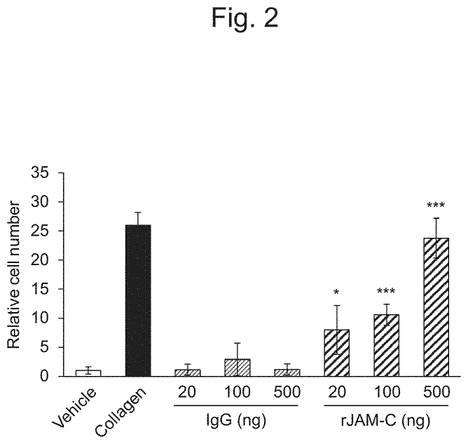

First, the adhesiveness of recombinant soluble JAM-C (recombinant JAM-C (hereinafter also referred to as rJAM-C)) with ADSCs was evaluated. After rJAM-C or IgG as a control was added to a plastic culture dish, ADSCs were added and 30 min later the cells were stained with crystal violet and the adherent cell number was counted. In the case of rJAM-C the adhesion was significantly increased at 20, 100, and 500 ng (FIG. 2). These results showed that extracellular rJAM-C strongly bound to ADSCs.

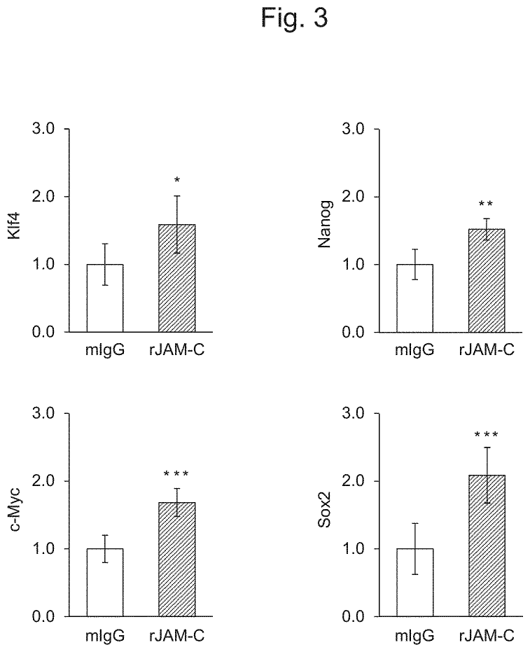

Next, ADSCs were cultured in a culture dish coated with rJAM-C, and the expression of stem cell markers Klf4, Nanog, c-Myc, and Sox2 were evaluated by semi-quantitative RT-PCR. The results showed that the expression of all four stem cell markers tested was significantly increased in ADSCs cultured in the culture dish coated with rJAM-C (FIG. 3).

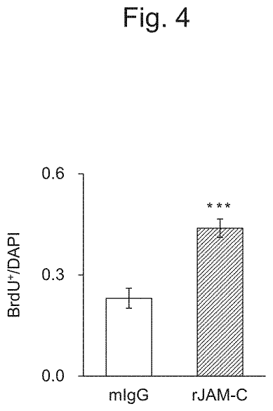

Next, ADSCs were cultured in a culture dish coated with rJAM-C, and the nuclei of cells in proliferation phase were labeled with BrdU, which were divided by the number of DAPI-positive cells to evaluate cell proliferation. The results showed that the proliferation ability was significantly increased in the ADSCs cultured on rJAM-C as high as about 2 fold (FIG. 4).

These results revealed that sJAM-C deposited on the substrate functioned as a stem cell niche and contributed to the proliferation and the maintenance of stem cell potential of ADSCs.

### Example 3: Preparation of Peptide

Chemical synthesis and purification of the JAM-C D1 peptide (hereinafter also referred to as JAM-C D1) consisting of the amino acid sequence of DPRIEWKKI (SEQ ID NO: 33) was entrusted to Eurofins, Inc.

Liquid chromatography was performed under the following conditions.

Column: YMC-Pack ODS-AM φ4.6 mm×75 mm
Mobile phase: A (0.02% TFA in water) and B (0.02% TFA in acetonitrile)
Flow rate: 1.0000 mL/min

### Detection: 214 nm

Column oven temperature: 40° C.

The purity of the peptide estimated from the peak area was 98.145%.

Subsequently, MS was performed under the following conditions.

### Probe: ESI

Gas flow rate: 1.5 L/min
DL temperature: 250° C.
Block temperature: 200° C.
Probe bias: +1.10 kV

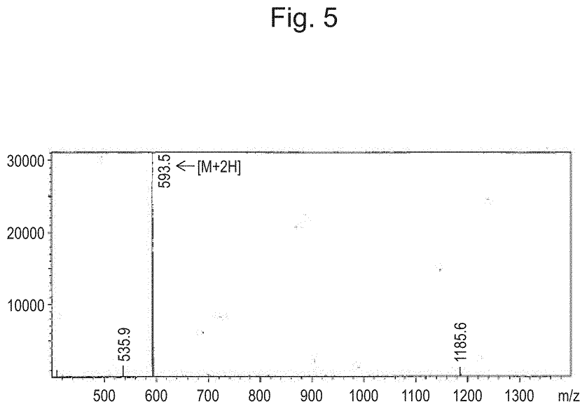

The resulted average MS spectrum is shown in FIG. 5.

### Example 4: Effect of the Peptide on Cells

### (Material and Method)

An experiment was conducted basically in the same manner as in Example 2, except that the JAM-C D1 prepared in Example 3 was used, and F9 cells expressing JAM-B and JAM-C(Satohisa, et al., Exp. Cell Res., 2005, 310, 66-78) were used instead of ADSCs in the adhesion test.

### (Results)

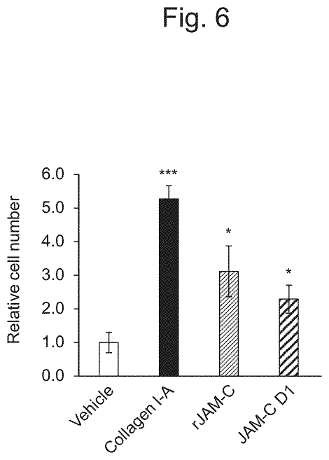

The results of evaluation of the adhesiveness of the JAM-C D1 peptide with the F9 cells are shown in FIG. 6. As shown in FIG. 6, the adhesion was significantly increased by JAM-C D1. These results showed that the JAM-C D1 strongly bound to the F9 cells.

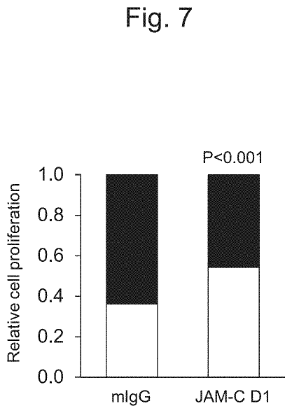

The results of evaluation of cell proliferation using BrdU staining and DAPI staining are shown in FIG. 7. The white column shows the BrdU-positive cell number divided by the DAPI-positive cell number, and the black column shows the BrdU-negative cell number divided by the DAPI-positive cell number. As shown in FIG. 7, the proliferation ability of the ADSCs was significantly increased by the JAM-C D1.

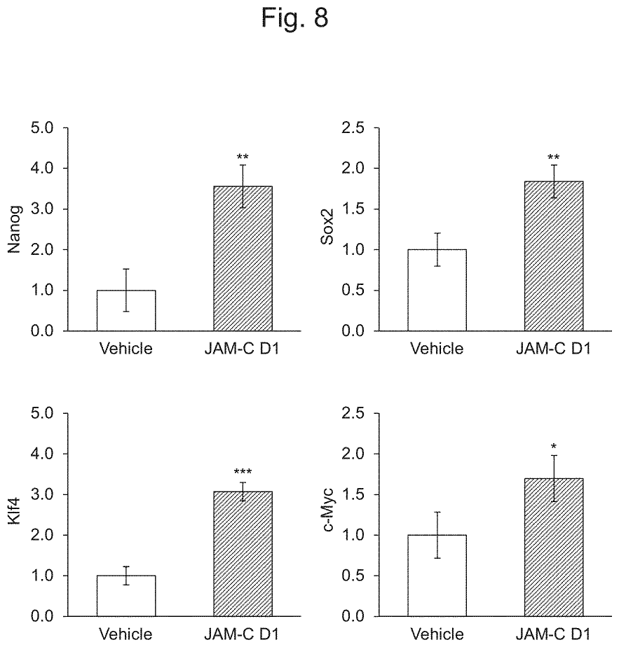

FIG. 8 shows the results for which ADSCs were cultured in a culture dish coated with JAM-C D1 and the expressions of stem cell markers Nanog, Klf4, Sox2, and c-Myc were evaluated by semi-quantitative RT-PCR. The results showed that the expression of all four kinds of stem cell markers tested was significantly increased in the ADSCs cultured in the culture dish coated with JAM-C D1.

All publications, patents, and patent applications cited herein are directly incorporated herein by reference.

1. A peptide comprising DPRIEWKKI (SEQ ID NO: 33), or an amino acid sequence obtained by adding, deleting, and/or substituting one amino acid in the amino acid sequence of SEQ ID NO: 33, or a salt thereof.

2. A coating agent for a cell culture substrate comprising the peptide or salt thereof according to claim 1.

3. A cell culture substrate coated with the peptide or salt thereof according to claim 1.

4. (canceled)

5. An agent for promoting cell proliferation comprising the peptide or salt thereof according to claim 1.

6. An agent for maintaining stem cell potential comprising the peptide or salt thereof according to claim 1.

7. A culture medium comprising the peptide or salt thereof according to claim 1.

8. (canceled)

9. A method of culturing a cell comprising:
a step of coating a cell culture substrate with a peptide comprising DPRIEWKKI (SEQ ID NO: 33), or an amino acid sequence obtained by adding, deleting, and/or substituting one amino acid in the amino acid sequence of SEQ ID NO: 33, or a salt thereof, or the coating agent according to claim 2; and
a step of culturing a cell on the coated cell culture substrate.

10. A method of culturing a cell comprising a step of culturing a cell on the cell culture substrate according to claim 3.

11. A method of culturing a cell comprising a step of culturing a cell in the culture medium according to claim 7.

12. The method according to claim 9, which is a method of culturing a stem cell.

13. A method of producing a cell culture substrate comprising a step of coating a cell culture substrate with the coating agent according to claim 2.


SEQUENCE LISTING (33 sequences)

SEQ ID NO: 1 - protein, 310 aa, Homo sapiens
MALRRPPRLRLCARLPDFFLLLLFRGCLIGAVNLKSSNRTPVVQEFESVELSCIITDSQTSDPRIEWKKIQDEQTTYVFFDNKIQGDLAGRAEILGKTSLKIWNVTRRDSALYRCEVVARNDRKEIDEIVIELTVQVKPVTPVCRVPKAVPVGKMATLHCQESEGHPRPHYSWYRNDVPLPTDSRANPRFRNSSFHLNSETGTLVFTAVHKDDSGQYYCIASNDAGSARCEEQEMEVYDLNIGGIIGGVLVVLAVLALITLGICCAYRRGYFINNKQDGESYKNPGKPDGVNYIRTDEEGDFRHKSSFVI

SEQ ID NO: 2 - DNA, 933 bp, Homo sapiens
atggcgctgaggcggccaccgcgactccggctctgcgctcggctgcctgacttcttcctgctgctgcttttcaggggctgcctgataggggctgtaaatctcaaatccagcaatcgaaccccagtggtacaggaatttgaaagtgtggaactgtcttgcatcattacggattcgcagacaagtgaccccaggatcgagtggaagaaaattcaagatgaacaaaccacatatgtgttttttgacaacaaaattcagggagacttggcgggtcgtgcagaaatactggggaagacatccctgaagatctggaatgtgacacggagagactcagccctttatcgctgtgaggtcgttgctcgaaatgaccgcaaggaaattgatgagattgtgatcgagttaactgtgcaagtgaagccagtgacccctgtctgtagagtgccgaaggctgtaccagtaggcaagatggcaacactgcactgccaggagagtgagggccacccccggcctcactacagctggtatcgcaatgatgtaccactgcccacggattccagagccaatcccagatttcgcaattcttctttccacttaaactctgaaacaggcactttggtgttcactgctgttcacaaggacgactctgggcagtactactgcattgcttccaatgacgcaggctcagccaggtgtgaggagcaggagatggaagtctatgacctgaacattggcggaattattgggggggttctggttgtccttgctgtactggccctgatcacgttgggcatctgctgtgcatacagacgtggctacttcatcaacaataaacaggatggagaaagttacaagaacccagggaaaccagatggagttaactacatccgcactgacgaggagggcgacttcagacacaagtcatcgtttgtgatctga

SEQ ID NO: 3 - protein, 310 aa, Macaca mulatta
MALRRPPRLRLCARLPDFVLLLLFRGCLIGAVNLKSSNRTPVVQEFESVELSCIITDSQTSDPRIEWKKIQDDQTTYVFFDNKIQGDLAGRAEILGKTSLKIWNVTRRDSALYRCEVVARNDRKEIDEIVIELTVQVKPVTPVCRVPKAVPVGKMATLYCQESEGHPRPHYSWYRNDVPLPTDSRANPRFRNSSFHLNSETGTLVFTAVHKDDSGQYYCIASNDAGSARCEEQEMEVYDLNIGGIIGGVLVVLAVLALITLGICCAYRRGYFINNKQDGESYKNPGKPDGVNYIRTDEEGDFRHKSSFVI

SEQ ID NO: 4 - DNA, 933 bp, Macaca mulatta
atggcgctgaggcggccgccgcgactccggctctgcgctcggctgcccgacttcgtcctgctgctgctgttcaggggctgcctgataggggctgtaaatctcaaatccagcaatcgaaccccagtggtacaggaatttgaaagtgtggaactgtcttgcatcattacagattcgcagacaagtgaccccaggatcgaatggaagaaaattcaagatgaccaaaccacatacgtgttttttgacaacaaaattcagggagacttggcaggtcgtgcagaaatactggggaagacgtccctgaagatctggaatgtgacacggagagactcggctctctatcgctgtgaggtggttgctcgaaatgaccgcaaggaaattgatgagattgtgatcgagttaactgtgcaagtgaagccagtgacccctgtctgtagagtgccgaaggctgtaccagtaggcaagatggcaacgctgtactgccaggagagtgagggccacccccggcctcactacagctggtatcgcaatgatgtgccactgcccacagattccagagccaatcccagatttcgcaattcttctttccacttaaactctgaaacaggcactctggtgttcactgctgttcacaaggacgactctgggcagtactactgcattgcttccaatgacgcaggctcagccaggtgtgaggagcaggagatggaagtctatgacctgaacattggcggaattattgggggggttctggttgtccttgctgtactggccctgatcacgttgggcatctgctgtgcatacagacgtggctacttcatcaacaataaacaggatggagaaagttacaagaacccagggaaaccagatggagtcaactacatccgcactgacgaggagggcgacttcagacacaagtcatcgtttgtgatctga

SEQ ID NO: 5 - protein, 317 aa, Canis familiaris
MALRRRPAGWLRARLPGLVLLLLLRGCLTGAVNLKSSNRTPVVQEFESVELSCIITDSQTTDPRIEWKKIQDDQTTYVFFDNRIQGDLAGRAELLGKTSLRIWNVTRTDSALYRCEVVARNDRKEIDEIVIELTVQVKPVSPVCRVPKAVPVGKTATLHCQESEGYPRPHYSWYRNDVPLPTDSRANPRFRNSSFLLNSETGTLVFSAVHKEDSGQYYCIASNDAGSARCEEQQMEVYDLNVGGIVGGILVVLAVLALITVGICCAYRRGYFVNNKQNGESPFSSFFSYKSPGKPDGVNYIRTDEEGDFRHKSSFVI

SEQ ID NO: 6 - DNA, 954 bp, Canis familiaris
atggcgctcaggcggcggccggcgggttggctccgcgcgcggctgcccggcctcgtcctcctgctgctgctcaggggttgcttgaccggggccgtgaatctcaagtccagcaaccggaccccagtggtccaggagtttgaaagtgtggaactatcctgtatcattacggattcacagacgactgaccccaggattgaatggaagaaaattcaagatgaccaaaccacatatgtgttttttgacaacagaattcagggagatttggcaggtcgtgcagaactattggggaagacctccctcaggatctggaacgtgacccggacagattcagccctttatcgctgtgaggtggttgctcgaaacgaccgcaaagaaattgatgagattgtcattgagttaactgtacaagtgaagccagtatctcctgtgtgcagagtcccgaaggctgtccctgtgggcaagacggccacgctgcactgccaggagagtgagggctacccccggccccactacagctggtaccgcaatgatgtgccgctgcccacagattccagagccaatcccagatttcgaaactcctcttttcttttaaactctgaaacaggcactctggttttcagtgctgttcacaaggaggactctgggcagtattactgcatcgcttccaatgacgcaggctcggccagatgtgaggagcagcagatggaagtctatgacctgaacgttggcggcatcgttggtgggatcctggtggtccttgccgtcctggccctgataacagtgggcatctgctgcgcatacagacggggttactttgtcaataataaacagaatggcgaaagccctttctcctcctttttcagttacaagagtccagggaagccagatggcgtgaactatatcaggacagatgaggagggcgacttcagacacaagtcatcatttgtcatctga

SEQ ID NO: 7 - protein, 310 aa, Mus musculus
MALSRRLRLRLYARLPDFFLLLLFRGCMIEAVNLKSSNRNPVVHEFESVELSCIITDSQTSDPRIEWKKIQDGQTTYVYFDNKIQGDLAGRTDVFGKTSLRIWNVTRSDSAIYRCEVVALNDRKEVDEITIELIVQVKPVTPVCRIPAAVPVGKTATLQCQESEGYPRPHYSWYRNDVPLPTDSRANPRFQNSSFHVNSETGTLVFNAVHKDDSGQYYCIASNDAGAARCEGQDMEVYDLNIAGIIGGVLVVLIVLAVITMGICCAYRRGCFISSKQDGESYKSPGKHDGVNYIRTSEEGDFRHKSSFVI

SEQ ID NO: 8 - DNA, 933 bp, Mus musculus
atggcgctgagccggcggctgcgacttcgactgtacgcgcggctgcctgacttcttcctgctgctgctcttcaggggctgcatgatagaggcagtgaatctcaaatccagcaaccgaaacccagtggtacatgaatttgaaagtgtggaattgtcttgcatcattacggactcacagacaagtgaccctaggattgaatggaagaaaatccaagatggccaaaccacatatgtgtattttgacaacaagattcaaggagacctggcaggtcgcacagatgtgtttggaaaaacttccctgaggatctggaatgtgacacgatcggattcagccatctatcgctgtgaggtcgttgctctaaatgaccgaaaagaagttgatgagattaccattgagttaattgtgcaagtgaagccagtgacccctgtctgcagaattccagccgctgtacctgtaggcaagacggcaacactgcagtgccaagagagcgagggctatccccggcctcactacagctggtaccgcaatgatgtgccactgcctacagattccagagccaatcccaggttccagaattcctctttccatgtgaactcggagacaggcactctggttttcaatgctgtccacaaggacgactctgggcagtactactgcattgcttccaatgacgcaggtgcagccaggtgtgaggggcaggacatggaagtctatgatttgaacattgctgggattattgggggagtccttgttgtccttattgttcttgctgtgattacgatgggcatctgctgtgcgtacagacgaggctgcttcatcagcagtaaacaagatggagaaagctataagagcccagggaagcatgacggtgttaactacatccggacgagtgaggagggtgacttcagacacaaatcgtcctttgttatctga

SEQ ID NO: 9 - protein, 310 aa, Rattus norvegicus
MALSRRLRLRLCARLPDFFLLLLFRGCVIEAVNLKSSNRNPVVHEFESVELSCIITDSQTNDPRIEWKKIQDGQTTYVYFDNKIQGDLAGRTDVFGKTSLRIWNVTRSDSAIYRCEVVALNDRKEVDELTIELIVQVKPVAPVCRVPKAVPVGKAATLQCQESEGYPRPYYSWYRNDVPLPTDSRANPRFQNSSFHVNSETGTLVFSAVHKEDSGQYYCIASNDAGAARCEGQDMEVYDLNIAGIIGGVLVVLIVLAVITMGICCAYRRGCFISSKQDGESYKSPGKHEGVNYIRTSEEGDFRHKSSFVI

SEQ ID NO: 10 - DNA, 933 bp, Rattus norvegicus
atggcgctgagccggcggctgcgacttcgcctgtgcgcgcggctacccgacttcttcctgctgctgctcttcaggggctgcgtgatagaggcagtgaacctcaaatccagcaaccgcaacccagtggttcatgaatttgaaagtgtggaattgtcttgtatcattacggactcacagacaaatgaccctaggattgaatggaagaaaattcaagatggccaaaccacatatgtgtattttgacaacaagattcaaggagacctggccggtcgcacagatgtgtttggaaaaacttccctgaggatctggaacgtgacacggtcagactcagccatctatcgctgtgaggttgttgctctaaatgaccgaaaagaagttgatgagcttaccattgagttaattgtgcaagtgaagccagtggcccctgtgtgcagagttccaaaggctgtgcctgttggcaaggcagcaacactgcaatgccaagagagcgagggctatcctcggccgtactacagctggtaccgcaatgatgtgccactgcctacggattccagagccaatcccaggttccagaattcctccttccacgtgaactcggagacaggcactctggttttcagtgctgtgcacaaggaagactctgggcaatattactgcattgcttccaatgacgcaggcgcagcccggtgtgaggggcaggacatggaagtctatgatttgaacattgctgggattattgggggagtcctggttgtccttattgttcttgctgtgatcacaatgggcatctgctgtgcgtacagacgaggctgcttcatcagcagtaaacaggatggagaaagttataaaagcccagggaagcatgaaggcgttaactacatccggacaagtgaagagggtgacttcagacacaagtcatcctttgttatctga

SEQ ID NO: 11 - protein, 329 aa, Ovis aries
MVTGAGAGPRAGRVRGERARGPSAALDMALRRRPSLVLLLLLVRGCMIRAVNLKSSNRTPVVQEFESVELSCIITDSQTNDPRIEWKKIQDEQTTYVFFDNKIQGDLTDRAELLGKTSLKIWNVTRTDSALYRCEVVARNDRKEIDEIVIELTVQVKPVAPVCRVPRAVPVGKAATLSCQEGEGFPRPHYSWYRNDVPLPTDSRANQRFRNSSFILNPETGTLVFSAVHKEDSGQYYCIASNDAGSARCEEQDMEVYDLNIGGIIGGVLVVLTVLALITGGICCAYRRGYFISHSRNGESYKNPGKPDGVNYIRTDEEGDFRHKSSFVI

SEQ ID NO: 12 - DNA, 990 bp, Ovis aries
atggtgaccggggcgggcgcaggaccgcgggcagggcgcgtgcgcggggagcgggcccgcggcccctcagctgcgctcgacatggcgctgaggcgaaggccctcgttggtcctgctgctgttgcttgtgcggggctgcatgatcagggctgtgaatctcaaatccagcaaccgaaccccagtggttcaggaatttgaaagtgtggaactatcctgtatcattacggattcacagacgaatgaccccaggattgaatggaagaagatccaggatgaacaaaccacctatgtgtttttcgacaacaagattcagggagacctgaccgaccgtgcagagctgctggggaagacgtctctgaagatctggaacgtgacgaggacagactcggccctttaccgctgcgaggtggttgcccggaacgaccgcaaggagatcgacgagatcgtcatcgagttgaccgtgcaagtgaagccggtggccccggtgtgcagggtgccaagggccgtgcctgtgggcaaggcagccaccctgtcctgccaggagggcgagggcttcccacggccgcactacagctggtaccgcaacgacgtgccactgcccacagactccagggccaaccagcgcttccgaaactcctctttcatcttaaaccctgagacgggcactctggtgttcagcgccgtccacaaggaagactcgggccagtactactgcattgcatccaacgacgcgggctcggcccgctgtgaggagcaggacatggaagtgtatgacctcaacatcggcggcatcatcgggggggtcctagtcgtgctgaccgtcctggccctcatcacagggggcatctgctgtgcctatagacgtggctacttcatcagccacagccggaacggggaaagctacaagaacccagggaagccagatggagttaactacatcaggacagatgaggagggcgacttcagacacaagtcctcgtttgtgatctag

SEQ ID NO: 13 - protein, 300 aa, Gallus gallus
MALRRPALLLLLPLLGCRLLAVELTSSNTKPVVQEFQSVELSCIIKSTVTPDPRIEWKKIRDGETSYVFFDNKMQGDFATRAEILSRTSLVIKNTTRMDTATYRCEVAAPSDTKTIDEINIQLTVQVKPVTPRCTVPKAVPVGKTASLHCHENEGFPKSTYSWYRNSEPLSPDTKSNAKFQNSSYSLNPTTGTLVFHAVHKGDTGRYSCIATNDAGFAKCEEQEMEVYDLNIGGIIGGVLVVVAVLVLITLGICCAYRRGYFANSKENGESYKTPAKPDGVNYIRTDDEGDFRHKSSFVI

SEQ ID NO: 14 - DNA, 903 bp, Gallus gallus
atggcgctccggcggccggcgctcctcctgctgctgcccctgctcggctgcagactcttggccgtggagctgacatccagcaacaccaaacccgtggtgcaggaattccagagtgttgagctgtcctgcatcattaaatcaaccgtaacaccggatcccagaatcgagtggaagaaaatccgagatggagaaacctcttatgtgtttttcgacaataaaatgcagggcgactttgcaactcgggcagaaattctgagccggacatcactggtgatcaaaaacaccacccggatggacacggccacgtaccgctgtgaagtggcagcaccttctgacaccaaaaccatagatgagataaatatccagcttacagtccaagtgaaacctgtgactccgagatgcacagtgcctaaagctgtacctgtgggtaagacagcctccctccactgccacgagaatgaaggtttccccaagtctacgtacagctggtatcgcaacagtgagcctttatcaccggacacaaaatcaaatgccaaattccagaattcatcctacagcttgaatcccaccacaggcactctggtttttcatgctgtgcacaaaggtgacacgggccgttactcctgcattgcaacaaacgatgctggctttgccaagtgtgaggagcaggagatggaagtctatgacctcaacattggtgggataatcggtggagtcctggtggtcgtggcagtgttggtgctcatcactcttggtatctgctgtgcctacaggaggggctactttgcaaacagtaaagagaatggggaaagctacaagactccagcaaaacctgatggtgttaactatatccggacagatgatgagggcgacttcaggcacaagtcttcatttgtcatataa

SEQ ID NO: 15 - protein, 300 aa, Xenopus tropicalis
MAERSVQLMLLLIQGCSILAVELSTSNQNPVVQEHQSVELSCIITSTKTNDPRIEWKKIKNDDADYVYFENHIQGDLKGRARIQSKSSLFIQNTSRTDNGKYRCEVAALGDDKKIAEIYIFLTVQVKPVIPQCRVPKAVPVGKSAVLHCQENEGYPSSVYRWYRNSEALPDDSKSTLKFQNSSFTLDPKSGTLTFTAVNKGDMGRYYCIASNDAGSAKCEEQELEVYDLNIGGIIGGILVVLLVLALITGGICCAYRKGYCTSSSRSSGQSYKNPAKPEGVNYMRTNDEGDFRHKSSFVI

SEQ ID NO: 16 - DNA, 903 bp, Xenopus tropicalis
atggcggaacggagtgtacaactaatgttattactcatccagggttgcagtattctggctgtagaactgagcaccagcaaccaaaatcctgtggtgcaagaacatcagagtgttgagctgtcctgtattattaccagcactaaaacaaatgatcccagaattgaatggaagaaaatcaagaatgatgatgcagattatgtatactttgaaaatcacatccaaggtgacctaaaaggcagagctcgcatacagtcaaaaagctctctctttattcaaaataccagccgaacagacaacggaaagtacagatgtgaagtggctgctcttggtgatgacaaaaagatagcagaaatttacatcttcttgactgtacaagtgaagccagtaattccgcaatgtagagtgcctaaagctgtcccagtgggcaaatctgctgtacttcattgtcaagagaatgagggttacccaagttctgtgtatcgctggtaccgcaacagtgaagctctacctgatgactcaaaatcaactttgaaattccagaactcatcctttacacttgaccctaaatcaggtactctgacatttactgcagtcaacaaaggtgacatgggacgttattattgcattgcaagtaatgatgctggctcggccaagtgtgaggaacaagagctggaagtctatgacttgaatattggtggcatcataggaggtattcttgttgtacttcttgtactggcactgatcacaggaggtatttgctgtgcatatcgtaaaggctactgtacaagcagcagtagatccagtgggcagagctacaagaacccagcaaaaccagaaggtgtcaattacatgaggacaaatgatgagggtgatttcagacacaaatcttcatttgtcatatga

SEQ ID NO: 17 - protein, 302 aa, Danio rerio
MALTPLACVLLLLSMQCYISTLAVLLKSTNSKPWVNEFESIELSCMIESITTTKPRIEWKKIKNGDPSYVYFDNQISGDLERRAKIREPATLVILNATRSDSADYRCEVTAPNDQKSFDEILISLTVRVKPVVPRCSVPKSIPVGKPAELHCLEDEGYPKSQYQWFRNKEEIPLDPKSSPKFFNSTYTLDGEMGTLKFSAVRKEDAGEYYCRAKNEAGISECGPQMMEVYDINIAGIILGVVVVVMVLLCITVGIFCAYKRGYFTSQKQTGNNYKPPAKGDGVDYVRTEDEGDFRHKSSFVI

SEQ ID NO: 18 - DNA, 909 bp, Danio rerio
atggcgttaacaccactggcttgcgttctgcttctcctttccatgcaatgttacatcagcaccttggcagtcctcctgaagtcgacgaattctaaaccgtgggtcaacgagtttgaatcaatcgagctgtcctgcatgatcgagtccatcaccaccactaaacccagaatagagtggaagaagattaagaatggagacccaagctatgtgtattttgataatcaaatatcaggtgacttggaaaggagagcaaaaattcgagagcctgcaaccctggtcattcttaatgccacaagatcggacagcgctgactaccgctgtgaggtcacagcacccaatgaccagaaatccttcgatgagatcttgatatcactcactgtacgagtgaagccagtcgtgcccagatgctctgtgcccaagtctatcccggtgggcaaaccagcagagctgcattgcctggaggatgaaggctacccaaaatctcaataccagtggttccgcaacaaggaggaaatcccactagatcccaagagcagcccaaagttcttcaactcaacttacaccctagacggggaaatgggcacgctgaaattcagtgcggtcaggaaggaggacgcgggagaatattactgcagagccaaaaatgaggctggcatctctgaatgtggaccgcagatgatggaagtttatgacatcaacattgccggcatcatcttgggagtcgtggtggtggtgatggtcctcttgtgtataacagtgggcatcttctgtgcctataaacgtggctacttcaccagccagaagcagacgggaaacaattataaacctccagcaaaaggagatggagtggactatgtcagaacagaggatgagggggatttcaggcacaaatcctcatttgtcatctga

SEQ ID NO: 19 - DNA, 41 bp, Artificial
  primer
gagaattctgcagcggccgccatggcgctgagccggcggct

SEQ ID NO: 20 - DNA, 81 bp, Artificial
  primer
ctgcatagtccgggacgtcatacggatagcccgcatagtcaggaacatcgtatgggtagataacaaaggacgatttgtgtc

SEQ ID NO: 21 - DNA, 79 bp, Artificial
  primer
atgacgtcccggactatgcaggatcctatccatatgacgttccagattacgctgctactaacttcagcctgctgaagca

SEQ ID NO: 22 - DNA, 40 bp, Artificial
  primer
gggagaggggcggatcctaggcaccgggcttgcgggtcat

SEQ ID NO: 23 - DNA, 23 bp, Artificial
  primer
tagagctagactccgggcgatga

SEQ ID NO: 24 - DNA, 23 bp, Artificial
  primer
ttgccttaaacaagaccacgaaa

SEQ ID NO: 25 - DNA, 28 bp, Artificial
  primer
tgacctaactcgaggaggagctggaatc

SEQ ID NO: 26 - DNA, 32 bp, Artificial
  primer
aagtttgaggcagttaaaattatggctgaagc

SEQ ID NO: 27 - DNA, 24 bp, Artificial
  primer
agggtctgctactgagatgctctg

SEQ ID NO: 28 - DNA, 24 bp, Artificial
  primer
caaccactggtttttctgccaccg

SEQ ID NO: 29 - DNA, 24 bp, Artificial
  primer
gcgaactcacacaggcgagaaacc

SEQ ID NO: 30 - DNA, 23 bp, Artificial
  primer
tcgcttcctcttcctccgacaca

SEQ ID NO: 31 - DNA, 20 bp, Artificial
  primer
accacagtccatgccatcac

SEQ ID NO: 32 - DNA, 20 bp, Artificial
  primer
tccaccaccctgttgctgta

SEQ ID NO: 33 - protein, 9 aa, Artificial
  synthetic
DPRIEWKKI